(19) United States
(12) Patent Application Publication
(10) Pub. No.: US 2015/0080761 A1
(43) Pub. Date: Mar. 19, 2015

(54) CONTROL CIRCUIT AND SURGICAL TOOL

(71) Applicants: Jeffrey R. Schwindt, Indianapolis, IN (US); Bryan T. Burney, McCordsville, IN (US)

(72) Inventors: Jeffrey R. Schwindt, Indianapolis, IN (US); Bryan T. Burney, McCordsville, IN (US)

(21) Appl. No.: 13/261,921

(22) PCT Filed: Dec. 20, 2011
(86) PCT No.: PCT/US2011/066293

§ 371 Date: Jun. 20, 2014

Related U.S. Application Data
(60) Provisional application No. 61/459,897, filed on Dec. 20, 2010.

(51) Int. Cl. A61B 10/02; G01N 15/14

(52) U.S. Cl. CPC A61B 10/0275; G01N 15/14
USPC 600/567

(57) ABSTRACT

A control circuit is provided for use with a surgical tool, illustratively a vacuum-assisted surgical tool. The vacuum-assisted surgical tool has an outer cannula for insertion into a body to a point adjacent to a mass to be examined, and a cutter device may be housed within the outer cannula. In one embodiment, various other surgical devices may be housed within a multi-purpose outer cannula.

A rinse or other liquid can be provided for assisting in the removal of the mass to be examined. Other desirable fluids and materials may also be provided and placed in communication with the surgical tool. A vacuum source may also be provided for assisting in the removal of the mass to be examined. A cabinet may house portions of the pneumatic circuit and control circuit.

### BACKGROUND OF THE INVENTION

The present invention relates to a surgical tool, and particularly to a surgical tool in combination with a control circuit, the control circuit being useful in the operation of an at least partially pneumatically powered surgical tool.

Similar control circuits and surgical tools have been disclosed in U.S. Patent Application No. 60/374,952, Ser. Nos. 10/936,395, 10/420,212, 10/420,296 (now U.S. Pat. No. 7,316,726), Ser. Nos. 10/420,197, 11/970,155 (now U.S. Pat. No. 7,625,425), Ser. No. 11/970,168 (now U.S. Pat. No. 7,799,116), Ser. Nos. 11/965,428, 10/461,315 (now U.S. Pat. No. 7,749,172), Ser. Nos. 12/886,347, 12/830,350, and Ser. No. 12/209,881, all of which are incorporated herein by reference.

### SUMMARY OF THE INVENTION

The present disclosure relates to one or more of the following features, elements or combinations thereof. A medical device is provided. Such a medical device incorporates a control circuit for use with a surgical tool, illustratively a vacuum-assisted surgical tool. The vacuum-assisted surgical tool has an outer cannula for insertion into a body to a point adjacent to a mass to be examined, and a cutter device may be housed within the outer cannula. In one embodiment, various other surgical devices may be housed within a multi-purpose outer cannula.

A rinse or (illustratively) saline solution can be provided for assisting in the removal of the mass to be examined. Other desirable fluids and materials may also be provided and placed in communication with the surgical tool. A vacuum source may also be provided for assisting in the removal of the mass to be examined. A cabinet may house portions of the pneumatic circuit and control circuit.

The surgical tool may be composed substantially of polymeric materials and can be used in conjunction with a Magnetic Resonance Imaging device. Portions of the surgical tool may be flexible, as well. Collectively, the surgical tool and control circuit may be referred to herein as a “platform.”

A method of performing a surgical procedure also provided. The method comprises the steps of identifying a mass to be resected, using a surgical tool to resect the mass, and directing the resected tissue through an inner cannula to a receptacle. The method may additionally include the steps of analyzing certain tissue characteristics and generating data based on those characteristics, and comparing the data to previously collected data. Feedback to a surgeon can be provided so that the surgeon can make decisions on whether to proceed with the resection.

The disclosed platform is founded on the well-established principle that precise surgical resection can lead to extremely accurate surgical procedures, and when applied to the treatment of cancer, can help to eradicate cancer. What is lacking in the medical industry, and what is proposed herein, is a platform that provides instant feedback and data comparison, allowing a surgeon to have prompt comparison to previously collected normal tissue data. This would give a surgical team a critical advantage in the resection of cancerous tumors: the ability to know, core-by-core, whether the tissue being excised exhibits traits of normal tissue, or potentially cancerous, abnormal tissue.

Using the platform disclosed herein, voluminous tissue characteristic, data can be generated that has never been analyzed or studied before. Unlike any prior device, the platform captures data related to tissue density, fibrousness or firmness, and cellular and nuclear content. This would provide additional pathological insight into the specific tumor, and the resultant data can be studied post-operatively.

Additional features of the disclosure will become apparent to those skilled in the art upon consideration of the following detailed description of preferred embodiments exemplifying the best mode of carrying out the invention as presently perceived.

### BRIEF DESCRIPTION OF THE DRAWINGS

FIG. 1 is a side oblique overhead perspective view of a surgical tool shown in a hand wand embodiment;

FIG. 2 is a front perspective view of the surgical tool shown in FIG. 1;

FIG. 3 is an enlarged view of the tip of the cannula associated with the surgical tool shown in FIGS. 1 and 2, wherein the inner cutter is recessed;

FIG. 4 is a front perspective view of the surgical tool, similar to that shown in FIG. 2, wherein the inner cutter is in the ⅓ advanced position;

FIG. 5 is an enlarged side view of the tip of the cannula, similar to that shown in FIG. 3, wherein the inner cutter is in the ⅓ advanced position;

FIG. 6 is a front perspective view of the surgical tool, similar to that shown in FIGS. 2 and 4, wherein the inner cutter is in the fully advanced position;

FIG. 7 is an enlarged side view of the tip of the cannula, similar to that shown in FIGS. 3 and 5, wherein the inner cutter is in the fully advanced position;

FIG. 8 is a perspective view of the surgical tool, showing the inner components of the surgical tool through its transparent outer walls;

FIG. 9 is a rear perspective view of the surgical tool showing the rear attachment and inner components of the surgical tool;

FIG. 10 is a perspective view of the console housing;

FIG. 11 is a top perspective view of the console housing with the top cover removed;

FIG. 12 is a circuit diagram showing one embodiment of the pneumatic circuit disclosed herein;

FIG. 13 is a circuit diagram showing another embodiment of the pneumatic circuit disclosed herein;

FIG. 14 is a graphical representation of a flow cytometer as disclosed herein;

FIG. 15 is a screen shot of the interactive display disclosed herein;

FIG. 16 is a cross-sectional view of a dual-lumen embodiment of the invention, as disclosed herein;

FIG. 17 shows top and side views of one embodiment of the cannula disclosed herein;

FIG. 18 is a perspective view of the front panel and control circuit box associated with one embodiment of the invention;

FIG. 19 is a rear perspective view of the control circuit box shown in FIG. 18;

FIG. 20 is a perspective view of the internal components of the compressor housing;

FIG. 21 is another perspective view of internal components of the compressor housing;

FIG. 22 is a perspective view of another embodiment of the surgical tool, showing a position indicator incorporated into the surgical tool;

FIG. 23 is another view of the surgical tool shown in FIG. 22 with the detachable outer cannula removed and the cutting cannula attached;

FIG. 24 is a plan view of the distal end of the surgical tool shown in FIG. 22;

FIG. 25 is a perspective view of one end of the rotary air motor;

FIG. 26 is a perspective view of a blade receiver housed inside the distal end of the surgical tool;

FIG. 27 is a front view of a receptacle for receiving tissue cores;

FIG. 28 is an exploded view of the receptacle shown in FIG. 27;

FIG. 29 is a perspective view of another embodiment of the present invention;

FIG. 30 is another perspective view of the embodiment of FIG. 29;

FIG. 31 is an exploded perspective view of the embodiment of FIG. 29;

FIG. 32 is a perspective view of the housing associated with the embodiment of FIG. 29;

FIG. 33 is a cross-sectional view of the housing of FIG. 32 taken along the line 33-33 of FIG. 32;

FIG. 34 is view of the hub associated with FIG. 32;

FIG. 35 is a perspective view of the end cap associated with FIG. 32;

FIG. 36 is another perspective view of the end cap associated with FIG. 32; and

FIG. 37 is a perspective view of one embodiment of the cutter advancer holder.

### DETAILED DESCRIPTION OF EMBODIMENTS OF THE INVENTION

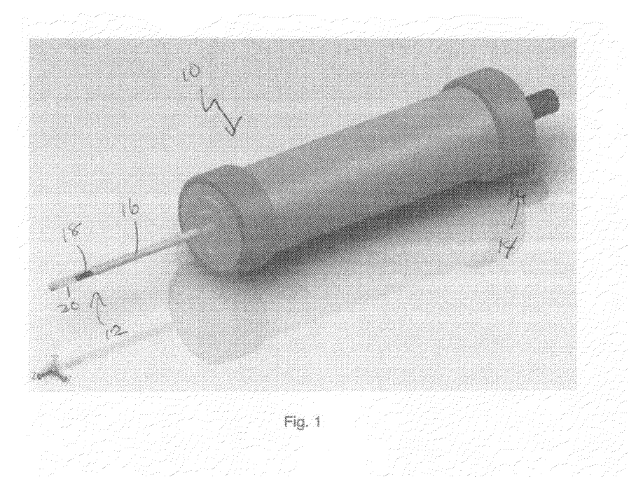

One aspect of the present disclosure is shown in FIG. 1, in the form of a surgical tool 10. Surgical tool 10 illustratively includes a distal end 12 that is configured to be inserted into a body, such as a living organism, and a proximal end 14 that is designed to be held by a surgeon or some type of positioning device. Such a surgical tool 10 may also be referred to as a hand wand, and the terms are considered synonymous herein.

The illustrated distal end 12 is sometimes referred to as a cannula within a cannula, or a tube within a tube. The distal end 12, in the illustrated embodiment, includes an outer cannula 16 and an inner cannula 18, visible in FIGS. 1-7, through an aperture 20 formed in outer cannula 16. Inner cannula 18 is illustratively a second tube, sharpened at its distal end that serves to cut tissue that is pulled by a vacuum through aperture 20 as the cannula 18 advances through outer cannula 16. However, it should be understood that other types of tools or devices may be substituted for inner cannula 18. For example, in another embodiment exemplified in FIG. 1B, a rotary blade may be substituted for inner cannula 18, wherein the rotary blade makes a lateral cut as the blade is rotating. In yet another embodiment, outer cannula 16 may house any number of alternative types of surgical or medical devices, such as brachytherapy, chemotherapy (e.g. chemotherapy beads), ablation devices, fluids, stains, cryogenic devices, cauterizing devices, lumens, cameras, fiber optics, lasers, balloons, catheters, surgical markers, and the like.

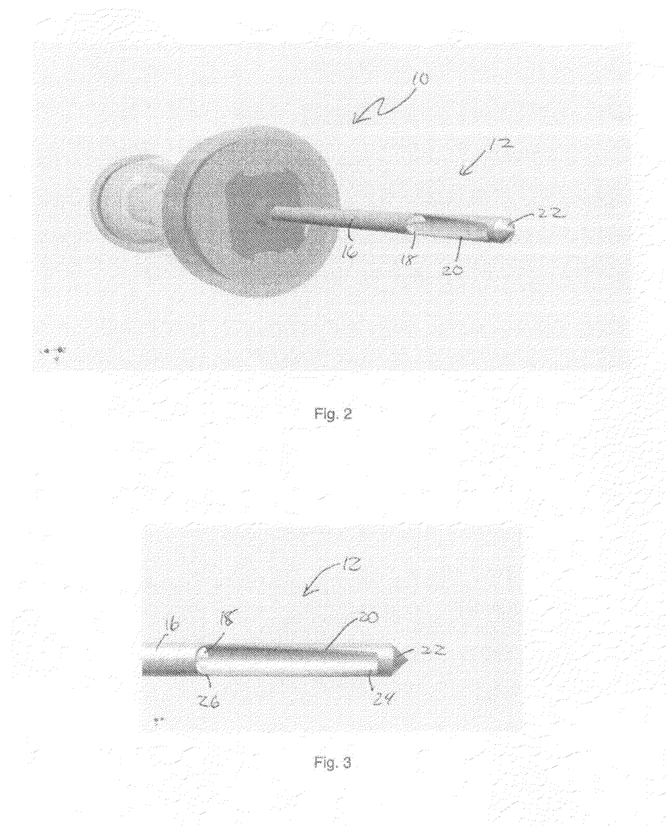

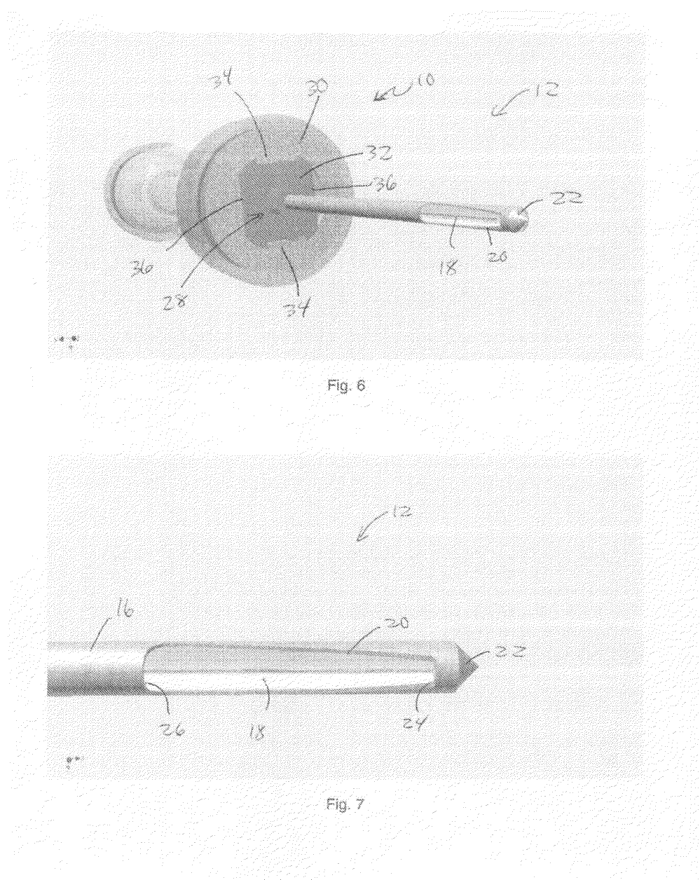

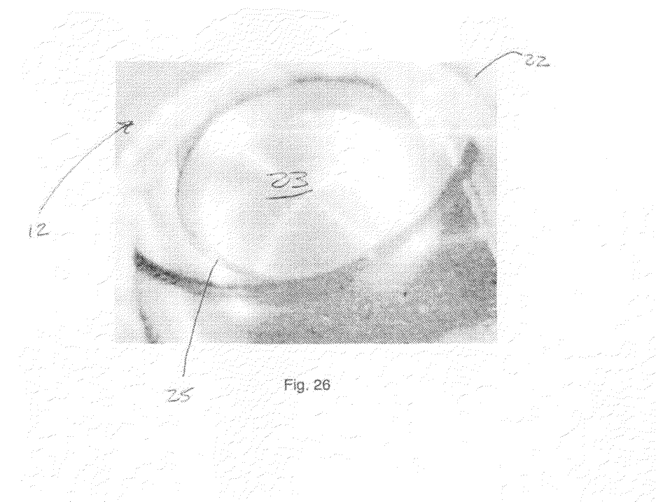

Turning to FIG. 2, the surgical tool 10 can be seen in perspective view from its distal end 12. Inner cannula 18, with its forward-leading cutter edge, can be seen positioned in outer cannula 16. A frusto-conical (illustratively stainless steel) tip 22 is shown press-fitted and/or laser welded on the end of outer cannula 16. Such a tip 22 may provide for easier insertion of the distal end 12 of surgical tool 10 into a patient's body. In another embodiment, a trocar tip may be substituted in place of the frusto-conical tip 22. A blade receiver 23, shown in FIG. 26, is illustratively housed within tip 22. Such a blade receiver may be used to enhance cutting, and is contemplated to be a selected shape or material that cooperates with inner cannula 18 during cutting. For example, blade receiver 23 may be formed of a plastic, metal, or other type of material that can serve as a cutting board mounted on the inside of tip 22, and/or may be a nipple 25 formed in tip 22, or a cylindrical rim positioned in tip 22 so as to contact and mate with inner cannula 18 when inner cannula 18 is at its full stroke position (the full stroke position being shown in FIGS. 6-7 and discussed in more detail herein).

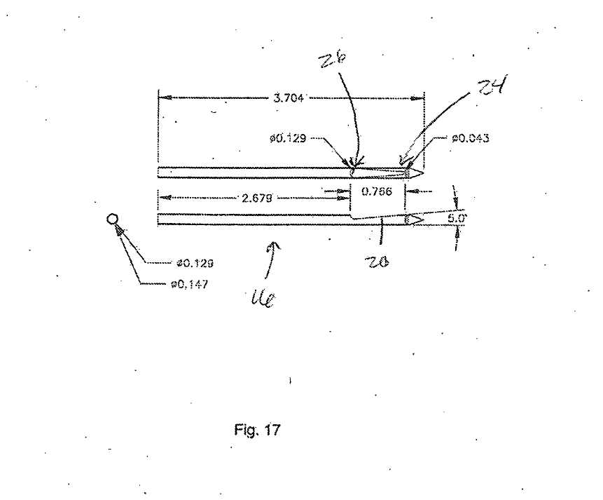

FIGS. 3 and 17 show various views of the distal end 12 of surgical tool 10. As can be seen in FIGS. 3 and 17, aperture 20 is formed such that distal end 24 of aperture 20 defines a width smaller than that of proximal end 26 of aperture 20. In such a configuration, distal end 24 assists with guiding inner cannula 18 so that inner cannula 18 does not impact or become wedged against the edge defined by the distal end 24 of aperture 20. Additionally, as discussed in detail below, such a configuration for aperture 20 permits “nibbling”—e.g. more precise resection of tissue masses. For example, in the illustrated embodiment, aperture 20 is tapered at a five-degree angle, as can be seen in FIG. 17.

In one embodiment (not shown), aperture 20 comprises a tapered edge that ramps inwardly, in a direction such that the sharp edge of the tapered edge is on the outer diameter of outer cannula 16. In such an embodiment, the sharp edge that is formed can serve as a cutting surface that directs tissue or other material inwardly toward the aperture. This embodiment also serves the purpose of directing inner cannula 18 away from edges of aperture 20 in the event inner cannula 18 is loosely held inside outer cannula 16. It should be understood that it may be desirable to have a loose fit between inner cannula 18 and outer cannula 16 so that friction between the two is minimized, and fluids can more easily pass between inner cannula 18 and outer cannula 16.

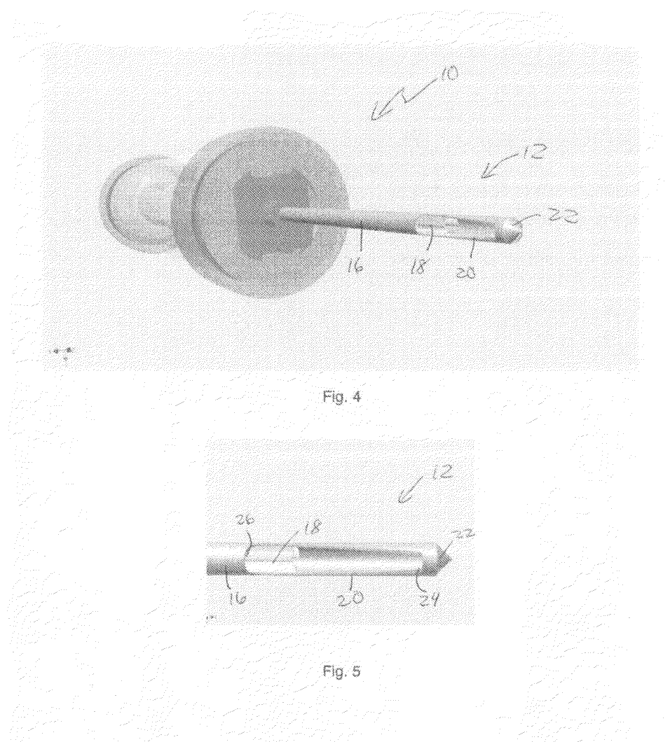

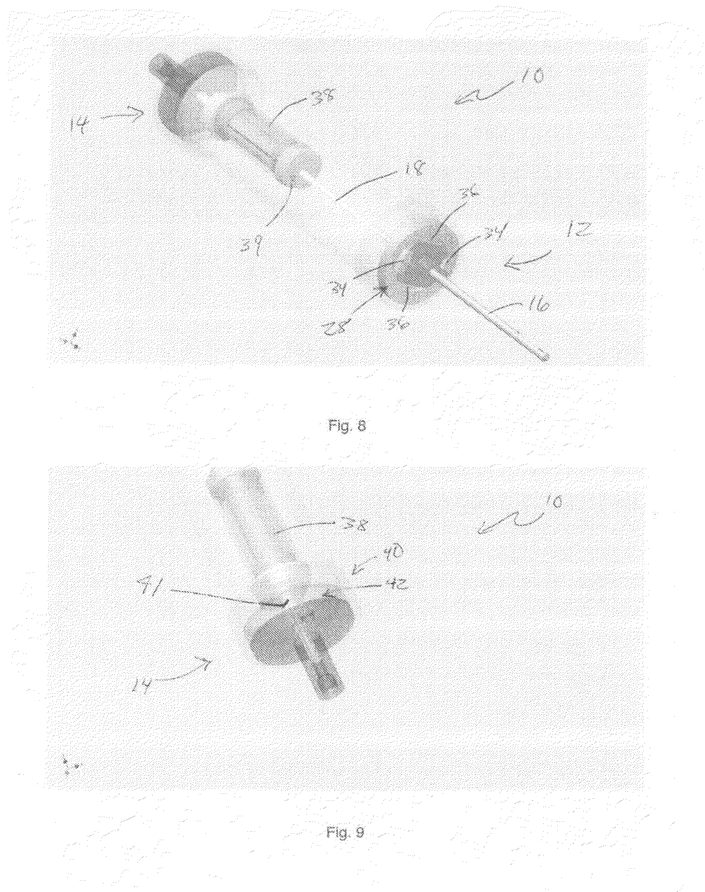

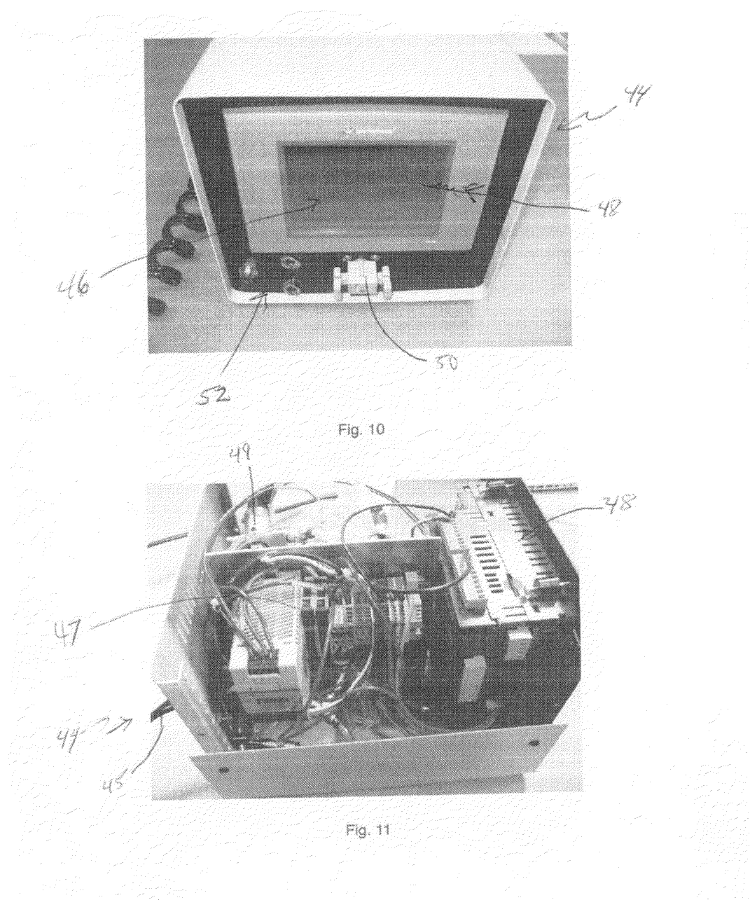

FIGS. 4 and 5 show surgical tool 10 in a position wherein the inner cannula 18 is partially advanced. Such an inner cannula 18 position is an intermediate position between the fully retracted position shown in FIGS. 2 and 3 and the fully extended position shown in FIGS. 6 and 7. In order to move inner cannula 18 between the fully retracted position of FIGS. 2-3 to the position shown in FIGS. 4-5, and also the fully extended position of FIGS. 6-7, a cutter advancer can be used. Such a cutter advancer is illustratively pneumatic, and is discussed in more detail below. Therefore, in the case of a pneumatic cutter advancer, by varying the amount of pneumatic pressure to the cutter advancer (shown in FIGS. 8-9 and discussed herein), a control circuit (shown in FIGS. 10-13 and 15 and discussed herein) can direct movement of inner cannula 18 relative to outer cannula 16.

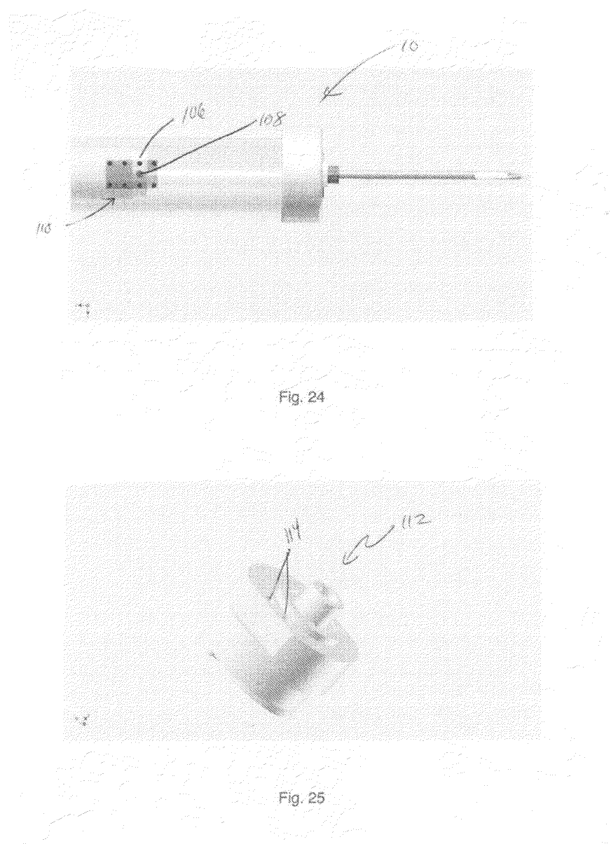

In the illustrated embodiment shown in FIGS. 1-9, outer cannula 16 is removably attached to surgical tool 10 via a hub 28. (FIG. 23 shows outer cannula 16 detached from surgical tool 10.) Having a detachable outer cannula 16 may be useful during surgical procedures that require additional steps after tissue resection. For example, a surgeon may wish to leave outer cannula 16 in a patient's body, yet remove the remainder of the surgical tool 10, so that outer cannula 16 yields a passageway to the surgical site. This may be desirable for the application of post-operative therapies such as inserting a marker, discussed further herein, or for post-operative procedures such as injecting pharmaceuticals, anesthetics, coagulants, brachytherapy, chemotherapy, fluids, or stains. It is also contemplated that cryogenic devices, cauterizing devices, lumens, cameras, fiber optics, lasers, balloons, catheters, surgical markers, ultrasound probes, prosthetics, tissue expanders, and the like can be inserted through outer cannula 16. Such treatments may have goals including any one or a combination of: medicating, inserting prosthetics, anesthetizing, viewing, radiating (e.g. brachytherapy), imaging, ablating, heating, cauterizing, stretching, freezing, coagulating, and marking with a locator.

As visible in FIGS. 6 and 23, hub 28 illustratively comprises a first member 30 and a second member 32 that is rotatable relative to first member 30. In the illustrated embodiment, in order to detach outer cannula 16, second member 32 is rotated 90 degrees counter-clockwise, such that wings 34 mounted on first member 30 align with spaces 36 formed in second member 32. This allows wings 34 to pass through spaces 36 as first member 30 is pulled away from second member 32. In another embodiment (not shown), wings 34 and spaces 36 may be unequally formed, so that each wing 34 is formed to mate with a specific space 36. This embodiment assures a specific registration of outer cannula relative to surgical tool 10, which would be advantageous to insure a known location of aperture 20 in relation to surgical tool 10. In other words, outer cannula only engages surgical tool 10 in one way.

It is also contemplated that one of the unequally formed wings 34 may be aligned with aperture 20, and thereby indicate the position of aperture 20 at the distal end 12 of outer cannula 16. This would assist a surgeon by providing a visual indication of the location of aperture 20, without requiring the removal of surgical tool 10 from the patient's body.

A perspective view of certain internal components of surgical tool 10 can be seen in FIGS. 8-9. In the illustrated embodiment, a rotary motor 38 is shown coupled to inner cannula 18. In the preferred embodiment, hollow inner cannula 18 serves a dual purpose: axle for rotary motor 38, and passageway through rotary motor 38, allowing fluids and excised tissue to travel through proximal end 14 of surgical tool 10.

Notably, rotary motor 38 is movable within surgical tool 10. Rotary motor 38 collectively moves with inner cannula 18 between a cannula-recessed position, shown in FIG. 8, and a cannula-extended position, shown in FIGS. 6-7. Advantageously, rotary motor 38 can be activated, and therefore cause inner cannula 18 to rotate, even as rotary motor 38 is moving between positions.

Rotary motor 38 is illustratively pneumatically powered, and a compressed air supply line can be directed through the surgical tool 10 housing to provide pneumatic power to activate rotary motor 38. The compressed air supply line is connected to input port 39, shown in FIG. 8.

In the illustrated embodiment, a pneumatic cutter advancer 40 is also positioned within surgical tool 10. In this embodiment, pneumatic cutter advancer 40 is an elastomeric member that stretches when activated by pneumatic pressure, and retracts when pneumatic pressure is released from surgical tool 10. However, it should be understood that cutter advancer 40 could be substituted by other types of devices that could function as discussed herein. For example, cutter advancer 40 could alternatively be replaced by a piston-type motor that reciprocates between an advanced position and a recessed position, as discussed herein or by a rolling diaphragm and a spring or by an accordion diaphragm and a spring.

Turning back to the illustrated configuration, pneumatic cutter advancer 40 is positioned toward the proximal end 14 of, and adjacent to, rotary motor 38 so that when it is activated, a portion of the pneumatic cutter advancer 40 acts on rotary motor 38 and causes it to move toward distal end 12 of surgical tool 10, thereby extending inner cannula 18. The elastomeric member may be formed of a variety of types of stretchy or elastomeric material, such as latex, Buna-N(Nitrile), silicone, or EDPM, or other synthetic rubbers. In certain applications, it may be advantageous to use a material that is hypoallergenic.

In the retraction stage, the cutter advancer may be considered to be acting as a return spring. An appropriate elastomeric member thickness would allow for both the extension and retraction of the rotary motor 38 when a selected pneumatic pressure is applied and reduced, respectively. In the embodiments disclosed herein, an appropriate thickness may range from 0.030 to 0.070 inch, or approximately 0.050 inch.

Cutter advancer 40 also functions as an anti-rotation mechanism for surgical tool 10. In particular, cutter advancer 40 is composed of such a material and constructed such that it inhibits rotation of rotary motor 38 within surgical tool 10 by virtue of its connection to rotary motor 38. In the illustrated embodiment, cutter advancer 40 includes a centrally located opening (not shown) in the elastomeric member that is stretched over a protrusion 41 (shown in FIG. 9) on the proximal end of rotary motor 38. Such an opening on the elastomeric member could be secured with a fastener.

In one embodiment, pneumatic cutter advancer 40 is connected to rotary motor 38 such that cutter advancer 40 both extends and retracts rotary motor 38 based on the amount of pneumatic pressure directed into cutter advancer 40. As can be seen in FIG. 9, when there is little to no back pressure (e.g. compressed air) directed into chamber 42 behind cutter advancer 40, cutter advancer 40 is in its retracted position. However, when compressed air begins to be directed into chamber 42, cutter advancer 40 begins to activate, thereby acting on rotary motor 38. Such pneumatic pressure, e.g. compressed air, directed into chamber 42 causes the elastomeric member to stretch accordingly.

Once the desired position of rotary motor 38, and therefore inner cannula 18, is achieved, pneumatic pressure into chamber 42 can be held constant, so as to hold rotary motor 38 and inner cannula 18 in place. This pressure may be held, for example, such that the inner cannula is in the fully extended position, or such that the inner cannula is in a position between fully extended and fully retracted—e.g. providing a partial aperture opening. While not required, temporarily holding inner cannula 18 at the fully extended position may be advantageous in a cutting stroke so that inner cannula 18 can continue to cut tissue while continuing to rotate even though not advancing at the moment and more effectively sever it from a patient's body. The inner cannula 18 may also be held in a certain position so that other steps of the surgical procedure may be performed, for example, moving the cut tissue core through surgical tool 10 with the use of saline and vacuum pressure.

Surgical tool 10 may be disposable, or may alternatively be used repeatedly in certain applications. In the embodiments contemplated herein, surgical tool 10 is connected via pneumatic tubing to a control circuit 44 that is configured to control the operation of surgical tool 10. The components comprising control circuit 44, and their associated functions in the control of surgical tool 10, are described below.

An exemplary front panel 46 of control circuit 44 is shown in FIG. 10. In the illustrated embodiment, front panel 46 includes a programmable interface 48 (e.g. with touch-screen capabilities), a pinch valve 50, and pneumatic connectors 52. Pinch valve 50 may function, for example, to control the flow of saline or other fluid into surgical tool 10. In such an embodiment, a saline feed line, e.g. a silicone tube, may be inserted between the central cantilevered catch and opposing cantilevered catches of pinch valve 50. When pulled taut, the silicone tubing would assume a substantially straight configuration and be disposed under the cantilevered catches. Such a configuration secures the silicone tubing and prevents accidental removal of silicone tubing from pinch valve 50. It is contemplated that other fluids, such as saline/anesthetic/vasoconstrictor combinations, may be routed through the saline feed line shown herein. This embodiment would provide convenience to a surgeon, since he would have less fluids to control, and would provide pain relief to a patient without separate injection, the pain relief hereby being administered directly into the wound ultimately exiting through the cutting notch into the patient. The amount of saline/anesthetic/vasoconstrictor solution that is injected into surgical tool 10 can also be modified by adjusting the amount of solution injected, and/or by adjusting the time between cutting strokes for the surgical tool 10 or by adjusting the dilution of the pharmaceutical in the fluid reservoir bag, or alternatively injecting said pharmaceutical through a one way valve side port (not shown) in the fluid tubing.

A pneumatically actuated stopper, not shown, is housed within pinch valve 50 and can be moved between a stopped position and a flow position. During operation, the default position for the pneumatically actuated stopper is the stopped position, stopping the flow of fluid through the silicone tubing. A plurality of pinch valves may be used in applications in which multiple fluids are delivered to surgical tool 10. Pinch valve(s) 50 may alternatively be positioned in other locations as desired.

Programmable interface 48 is illustratively an all-in-one unit that includes a processor, internal hard drive, SSD memory slot, a CANbus (illustratively two CANbuses), ethernet port(s), USB ports, and input and output ports. In the illustrated embodiment, a Unitronics® Vision570™ is used, which is described in more detail at http://www.unitronics.com/Series.aspx?Page=Vision570&ModelId=659. By using such an all-in-one unit, numerous procedures can be pre-programmed and various data may be recorded by a single display unit 48. Moreover, display unit 48 can illustratively include a color graphical user interface. Alternatively, it is contemplated that an inductive capacitance or motion recognition system may be used, such that actual touching is not required, but instead, the system can sense proximate movements from an attendant. This permits the system to be draped, as is often required during surgical procedures or controlled without drapes as the operator may remain sterile and control the unit without touching it. Accordingly, during operation, a user may simply touch or point toward portions of display unit 48 to set up the configuration of, and to control surgical tool 10.

An exemplary screen that may be programmed to be displayed by display unit 48 is shown in FIG. 15. Networked communication between display unit 48 and either a server or other computer is contemplated, such that monitoring and data acquisition may occur.

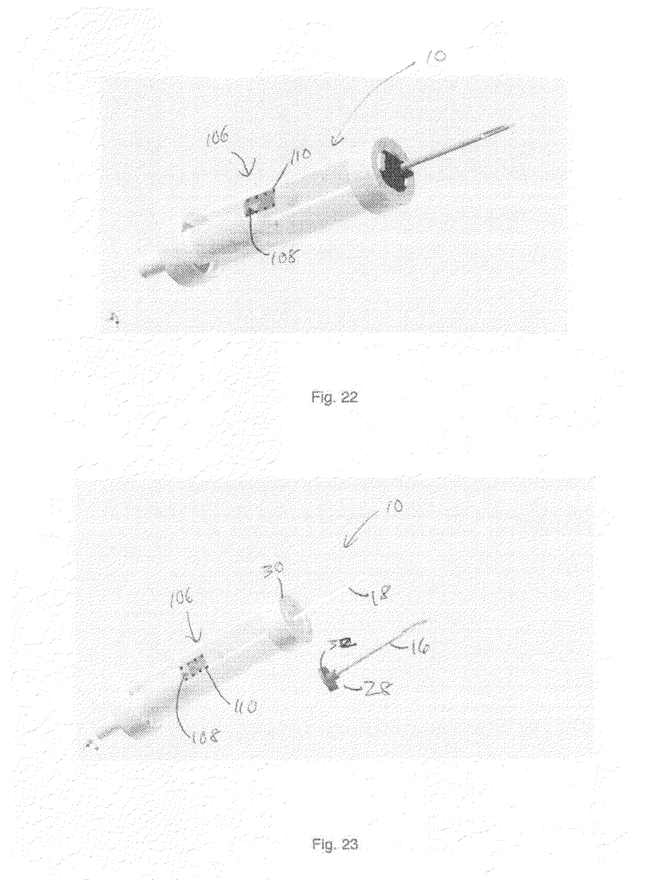

In yet another embodiment, shown in FIGS. 22-24, a visual indicator 106 is incorporated into surgical tool 10. In this embodiment, one end of rotary motor 38 has visible mark 108 that can be seen through a transparent or translucent portion 110 of surgical tool 10. Such a visual indicator 106 can provide indication to a surgeon as to how far inner cannula 18 is advanced relative to outer cannula 16. This would be helpful in knowing whether inner cannula 18 is fully extended, and whether retraction of inner cannula is full or some increment of full.

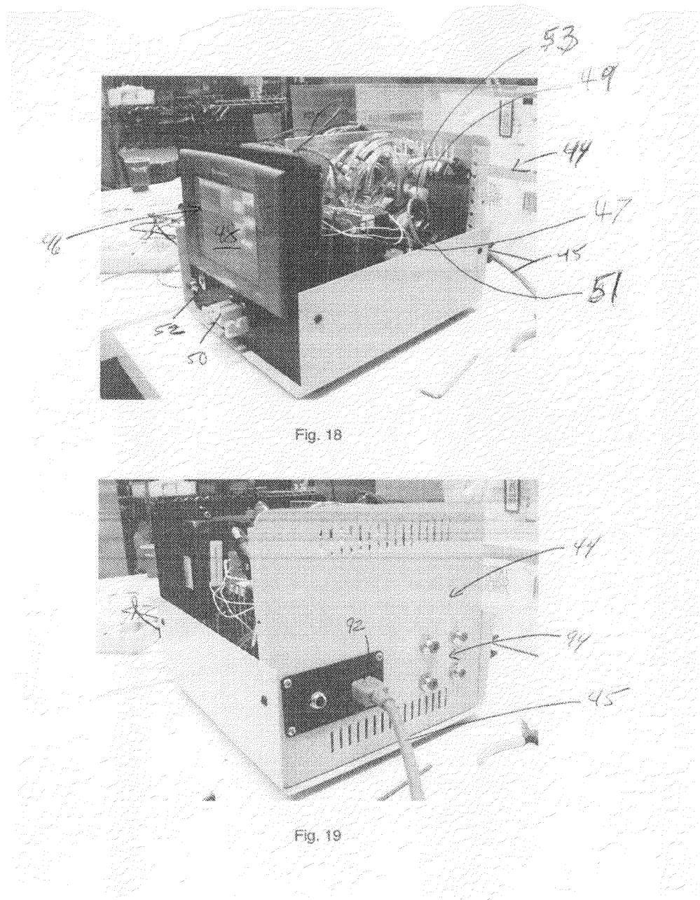

FIG. 11 shows control circuit 44 with its top cover removed. As can be seen in FIG. 11, display unit 48 may have various input and output ports, as well as other electronic connections and ports, on the back and sides of display unit 48. In the illustrated example, display unit 48 is connected to a power source 45, a valve bank 47, a pressure transducer 49, a vacuum transducer 51 (visible in FIG. 18), and a needle valve 53 (visible in FIG. 18).

In one embodiment, control circuit 44 is used to control a pneumatic circuit 54 such as that symbolized in the circuit diagram seen in FIG. 12. In the embodiment shown in FIG. 12, pneumatic circuit 54 includes a proportional regulator 56, a valve 58, a flow meter 60, an air motor 62, and a flow control 64. Air motor 62 may be, for example, the rotary motor 38 of surgical tool 10, described above. However, it should be understood that air motor 62 may alternatively be any other type of pneumatic motor or compressed air source that could be contemplated in a medical device.

The flow meter 60 and flow control 64 of pneumatic circuit 54 provide a method of monitoring and controlling the flow of compressed air through, air motor 62. This may be advantageous in applications in which a surgeon desires to know when air motor is hindered in some way by the material (e.g. tissue) it is cutting, or when it is desirable to increase or decrease the rotational speed of air motor 62. Additionally, this provides the advantage of determining more accurate characteristics (e.g. fibrousness or density) of the tissue or other material being cut.

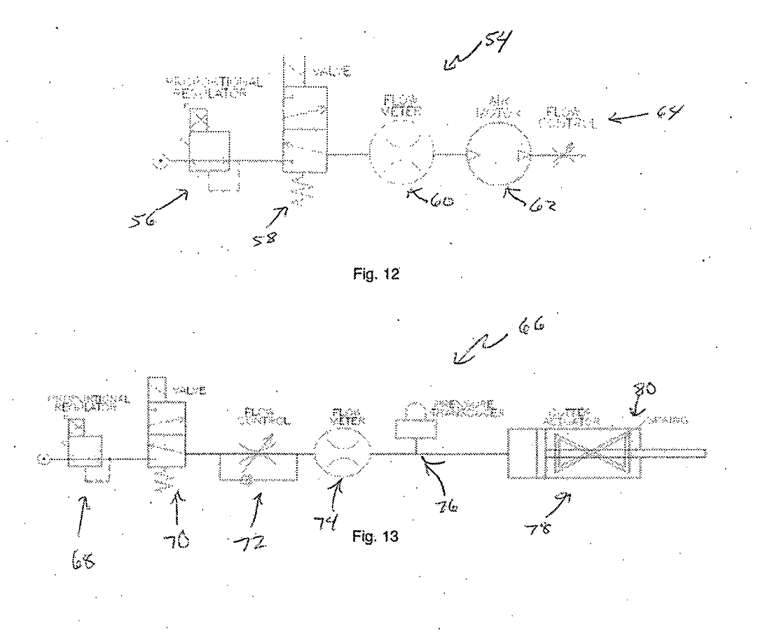

In another embodiment, a pneumatic circuit 66, shown in FIG. 13, is contemplated. In this embodiment, pneumatic circuit 66 includes a proportional regulator 68, valve 70, flow control 72, flow meter 74, pressure transducer 76, and a cutter actuator 78. Pneumatic circuit 66 may be used, for example, to control a cutter advancer 40 as described herein. In such an embodiment, cutter actuator 78 and spring 80 are replaced by the elastomeric cutter advancer 40 disclosed above. Pneumatic circuit 66 can then monitor and control the actuation of cutter advancer 40, and in the illustrated example, therefore monitor and control the advancement of inner cannula 18. By tracking the flow of compressed air into pneumatic circuit 66 in relation to time or in relation to the cycle being performed, additional information about tissue characteristics may be obtained. For example, additional information regarding fibrousness or density of the tissue being resected may be obtained. In the case of both pneumatic circuit 54 and pneumatic circuit 66, such tissue characteristics can be assigned numeric or other values, and the data stored by display unit 48. Such data may also be compared with data from other tissue resections and compared for similarities or norms. Reports on the comparison of the data to normal tissue data and/or abnormal tissue data may be reported by display unit 48 for immediate feedback to an attending surgeon. By using a flow meter 74, control circuit 44 can also determine the relative position of the inner cannula 18.

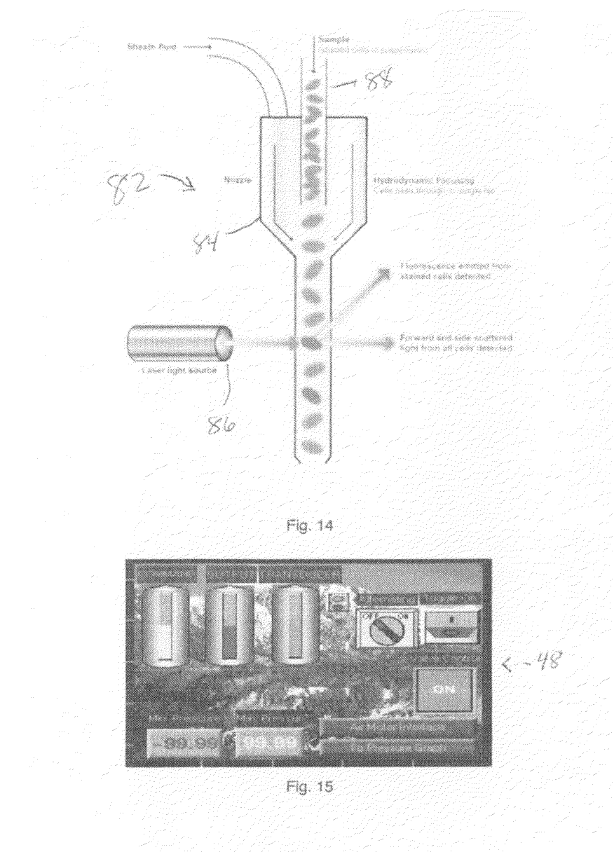

It is contemplated that surgical tool 10 may also be used in combination with a flow cytometer 82, such as that shown in FIG. 14. Such a flow cytometer 82 can be directly connected to surgical tool 10, or may be a separate component. In such an embodiment, additional data and characteristics relating to the resected tissue may be obtained. This additional data may be stored by display unit 48, compared to other data, and reported in conjunction (or separate from) the data captured and stored by pneumatic circuits 54 and 66. It is contemplated that tissue data such as that obtained by a flow cytometer can be useful in analyzing the nuclei of the cellular tissue. (See, for example, Thomas, Richard A., et al. “NASA/American Cancer Society High-Resolution Flow Cytometry Project-I.” Cytometry 43:2-11 (2001): 2-22, incorporated herein by reference.) Specifically, a flow cytometer can be used to compare the size of nuclei (and therefore the rate of division of the extracted cell nuclei) to a known normal value, providing a key perspective into whether tissue is abnormal or cancerous tissue. In the case of cellular tissue, a strong indicator of abnormal or cancerous tissue is whether there is a rapid rate of division. In the contemplated embodiment, a rate of division can be calculated within minutes of the tissue cell extraction (i.e. during the surgical procedure).

In the contemplated embodiment, a tissue sample or core would be directed toward flow cytometer 82 and the tissue cells separated, e.g. by being pulled apart or disrupted. The nibbling feature allows control of core sample thickness, thereby producing ideal size specimens for flow cytometry analysis. The cells could then be stained so that internal nuclei would be visible in flow cytometer. The cells are then introduced into the middle of a sheath fluid via conduit 88. A nozzle 84 is used to form a narrow stream of tissue and sheath fluid and thereby direct the stream past light source 86. Light source 86 emits laser beams that, when directed through the stream, refract depending on the tissue contained in the stream. In this way, nucleic content of the tissue may be monitored and analyzed.

In the embodiment shown in FIGS. 1-9, normal saline or other fluid (e.g. 5% dextrose in water) is introduced between outer cannula 16 and inner cannula 18 at distal end 12 of surgical tool 10. Saline fluid may be ported through the housing for surgical tool 10 for purposes such as rinsing the surgical site and assisting with moving a resected tissue core through the inner cannula and out through the proximal end 14 of surgical tool 10. By porting fluid through the housing of surgical tool 10, rather than outside of the surgical tool, it is not necessary to have a tube or line that attaches to hub 28. Accordingly, in the event hub 28 and outer cannula need to be removed during the procedure, the tube would not be left dangling from hub 28. This may be particularly advantageous for procedures in which multiple types of surgical tools are used, or when additional procedures are performed while leaving outer cannula 16 in position in the patient's body.

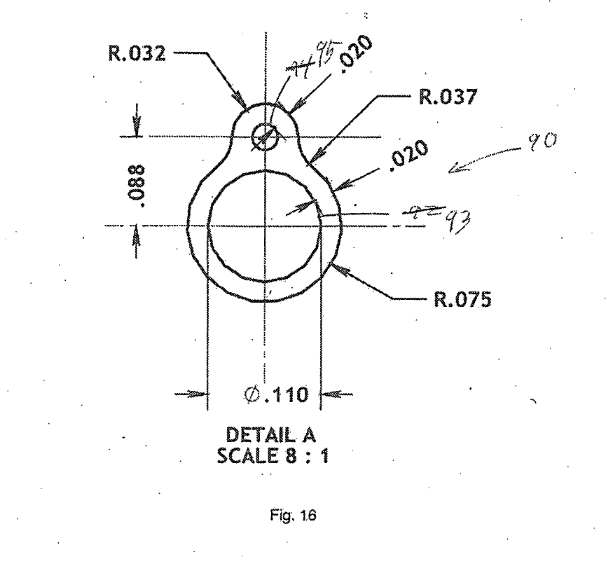

It may be advantageous in certain procedures to have a separate conduit for a secondary fluid, e.g. an anesthetic agent. In such a scenario, an alternative outer cannula 90 such as that shown in cross-sectional view in FIG. 16 may be used. In the illustrated embodiment, outer cannula 90 has as primary bore 93 for housing inner cannula 18, and a secondary bore 95 that may be used as a passageway for fluids, catheters, or other treatments. It is contemplated that secondary bore 95 may alternatively be used to house a fiber optic camera, a catheter, an ultrasonic probe, or any other type of surgical-assistive device. In the example of the fiber optic camera, the viewing end of the camera could be positioned inside outer cannula 90 such that it views tissue that is about to be cored through the aperture, when inner cannula 18 is retracted. In another embodiment, the viewing end of the camera may be positioned at the tip of outer cannula 90, such as in the trocar tip.

FIGS. 18-19 show additional views of the control circuit 44 and its housing. In FIG. 18, a perspective view of the front panel 46 of the control circuit can be seen. FIG. 19 shows a rear perspective view of the control circuit, illustrating the electrical connection 92, and pneumatic connections 94. Illustratively, two of the pneumatic connections 94 connect to a compressor and vacuum source. Other connections 94 may be used for connecting to a foot switch, toggle button (shown as element 16 in FIG. 29), or button on the surgical device 10, as discussed herein. It is contemplated that the button on the surgical device 10 may be, for example, a low-pressure air bulb that sends a signal (via air pressure) back to control circuit 44 via another tube. In an alternative embodiment, a manual valve inside surgical device 10 could be used to send a pressure signal back to control circuit 44.

In this scenario, it may not be necessary to have display unit 48 adjacent to the surgeon during a surgical procedure. Rather, the foot switch, toggle button, or surgical device button may be connected to control circuit 44 via a tube set (not shown). It is contemplated that such a tube set could be long enough to reach into a separate procedure room, MRI room, or the like. Moreover, if desired, display unit 48 could have a video output connection that sends video signals to an external monitor or the like (not shown). In another embodiment (not shown), the system may be activated by a button positioned on surgical tool 10.

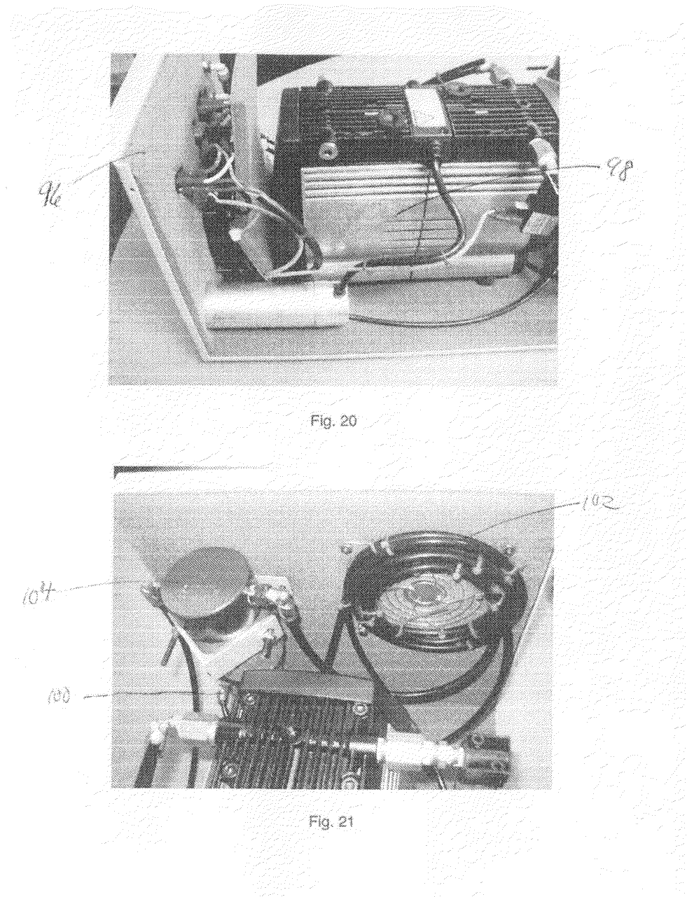

A compressor housing 96 is shown in FIGS. 20-21. In the illustrated embodiment, a compressor/vacuum assembly 98 is housed within compressor housing 96. The compressor/vacuum assembly is illustratively a combination compressor/vacuum, as can be obtained from Jun-Air at www.jun-air.com. However, it should be understood that other configurations and supplies for vacuum pressure and compressed air can be used. For example, it may be possible to have separate compressor and vacuum units. In another embodiment, portable compressed air may be used, such as can be obtained in compressed air tanks (disposable and/or refillable). Additionally, vacuum may be obtained via vacuum tanks, a hospital vacuum line, or other types of sources known to those skilled in the medical profession.

Compressor housing 96 may be incorporated with control circuit 44, or may be a separate unit, as shown in the illustrated embodiment. In the alternative, it is contemplated that certain facilities may have a centrally located compressor and vacuum system that can be accessed from numerous ports or locations.

In the embodiment shown in FIG. 21, a vortex cooling tube 100 is incorporated into the system—and illustratively positioned in compressor housing 96. Vortex cooling tube 100 uses the vortex effect to generate cold air, as described, for example, at http://vortec.com/vortex_tubes.php (incorporated herein by reference). By design, vortex cooling tubes typically use a great amount of compressed air flow to generate the cooling effect. However, in the pneumatic system disclosed herein, compressed air is being dumped from the system anyway. Accordingly, such compressed air is a candidate for redirection and use in a vortex tube.

In another embodiment, not shown, cooling can be established by using an electrical cooling system, such as a Peltier cooling system. An exemplary Peltier cooling system can be found at http://www.electronickits.com/kit/complete/peltier/ck501.htm, incorporated herein by reference.

Turning back to the illustrated embodiment, in addition to vortex cooling tube 100, a heat exchanger 102 is shown in FIG. 21. After vortex cooling tube 100 expels the cold air described above, a silicone tube or other conduit can direct the cold air toward heat exchanger 102. Heat exchanger 102 may be comprised of a dual-channel silicone tube or any other means suitable for exchanging heat between the cold air and hot compressed air coming from compressor vacuum assembly 98. By cooling the compressed air coming from compressor vacuum assembly 98, moisture may be more easily removed from the system. The resultant dry, compressed air is ideal for use in the pneumatic control system. A muffler, foam walls, and/or noise-canceling technology may be used in compressor housing 96 so as to reduce compressor and vacuum noise.

After passing through heat exchanger 102, cold air is directed toward filter housing 104, illustratively a coalescing filter. In one embodiment, the cold air output exiting from heat exchanger 102 is also used to cool other components in the compressor housing, such as compressor vacuum assembly 98. Furthermore, the dumping of cold air inside compressor housing 96 may also serve to reduce the inner housing temperature. Vortex cooling tube 100 also includes a hot exhaust, which can be used to vaporize any liquid moisture from the system.

To better illustrate the function of the surgical tool 10 and accompanying control circuit 44, a typical procedure will be described. A patient having a mass to be removed receives a local anesthetic and the mass is identified and located in the patient. Location methods may include physical examination, mammography, ultrasound, magnetic resonance imaging (MRI), X-Ray, or any other method known in the medical industry. Once surgical tool 10 has been connected to the control circuit, primed (including actuating cutter advancer 40), and inserted in a patient's body (illustratively adjacent to the tissue mass), a foot switch or other triggering device can be activated. The pneumatic signal from the triggering device will be sensed by control circuit 44. A vacuum valve is then energized, creating vacuum in a collection canister (not shown) and surgical tool 10. Display unit 48 then signals to direct compressed air to rotary motor 38.

Once a predetermined vacuum level is reached, inner cannula 18 can be retracted by reducing pneumatic pressure to cutter advancer 40. As discussed above, cutter advancer 40 is illustratively composed of such a material that it acts as a return spring, retracting inner cannula 18 when the pneumatic pressure is reduced. The full retraction of the inner cannula can be sensed by control circuit 44. However, it is contemplated that inner cannula 18 may be retracted to a point that is less than the full retraction, as discussed herein. For example, it may be desirable to retract inner cannula 18 only a third or two-thirds of the full distance. This retraction distance can be controlled by the amount of pressure maintained by control circuit 44 to pneumatic cutter advancer 40.

In another embodiment, cutter advancer 40 may comprise a rolling diaphragm and spring (not shown). The rolling diaphragm may be made of Buna N synthetic rubber or any other suitable material. In an alternative embodiment, the rolling diaphragm and/or spring may be interchangeable with other rolling diaphragms and springs so as to allow for the accommodation of various types of surgical tools. Still another alternative for cutter advancer 40 is a piston/bellows arrangement.

As inner cannula 18 is retracted, vacuum pressure (originating from compressor/vacuum assembly 98) causes tissue and/or other biological materials to be pulled inside inner cannula 18. As inner cannula 18 reaches the desired point of retraction (e.g. ⅓ retracted, ⅔ retracted, or fully retracted), rotary motor 38 can be activated by control circuit 44, so that rotary motor begins to rotate inner cannula 18. Fluid(s), such as saline, or lidocaine, may also be introduced at some point in the cycle. In the disclosed embodiment, saline is illustratively introduced during retraction of inner cannula 18, to assist with flushing biological materials through inner cannula 18.

Once inner cannula 18 is rotating, cutter advancer 40 can be activated by directing additional pneumatic pressure to cutter advancer 40. This causes inner cannula to advance. Tissue or other biological materials can be sucked inside aperture 20 via the vacuum pressure discussed herein, so that inner cannula 18 cuts the protruding tissue as inner cannula 18 advances through surgical tool 10.

Control circuit 44 may be configured to monitor the rotational speed of rotary motor 38, and may be additionally configured to monitor the back pressure in pneumatic cutter advancer 40. When abnormal readings are sensed, e.g. when rotation slows to an abnormal speed or cutter advancer 40 does not advance inner cannula 18 as expected, control circuit may be programmed to respond with additional compressed air to one or both of cutter advancer 40 and rotary motor 38. In addition, control circuit may direct less or more vacuum pressure to inner cannula 18. In yet another programming embodiment, control circuit 44 may instruct cutter advancer to retract a certain distance and begin the cutting stroke again. All of these options may be pre-programmed, or in the alternative, may be manually controlled by an operator.

In another control scheme, the processor could also be programmed to short stroke the cutter cylinder to “nibble” at the tissue when the monitored parameters indicate that a sample has not been taken. Such an action could be automatic and increase the efficiency of the device. It is also contemplated that a surgeon may wish to first rapidly de-bulk the tissue. Once the majority of the tumor is de-bulked, a surgeon may wish to “nibble” at the tumor margins, so that the surgeon more precisely removes the margins and does not remove any more tissue than required. Moreover, such tissue margins may be candidates for additional analysis, such as tissue characteristic or flow cytometer analysis, discussed further herein.

In the embodiments disclosed herein where tissue characteristics, flow cytometer analyses, or other data is generated, such data may be stored by display unit 48 in, for example, a flash drive. The data may also be compared to previously collected data for the particular patient, or for the general public. Display unit 48 may also report on any deviations from previous data or from the norm for the type of tissue being resected. Such data may enable a surgeon to determine whether additional resection is needed.

Once tissue and/or biological material has passed through inner cannula 18, it is directed toward the proximal end 14 of surgical tool 10 via vacuum pressure. In the illustrated embodiment, the biological and tissue material can then exit surgical tool 10 and is collected by a receptacle, for further analysis by a pathologist. The platform disclosed herein may also track each tissue core or biological material that passes through surgical tool 10, storing data for each cut and each core. Time and sequence in the surgical procedure may also be stored.

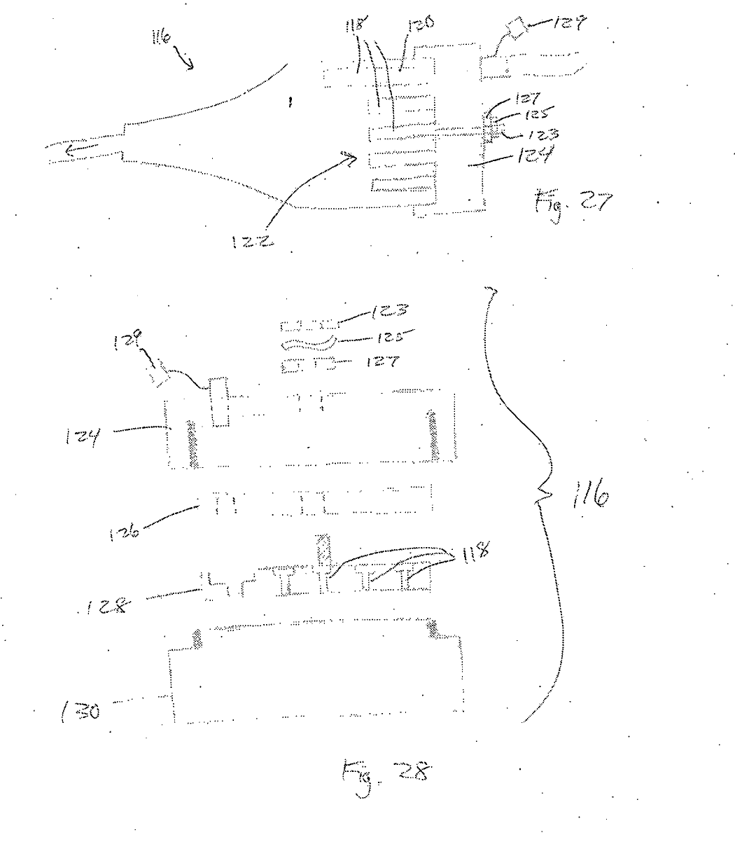

It is contemplated that the receptacle 116 could have a multi-chambered structure that receives tissue cores in a variety of chambers 118 within receptacle 116, such as that shown in FIGS. 27-28. For example, receptacle 116 may have a first chamber 120 (centrally or alternatively located) for collecting the majority of the tissue cores, such as during the tissue mass de-bulking stage. In this embodiment, receptacle 116 would also have a second chamber portion 122 with geographically assignable chambers.

In the disclosed embodiment, it is contemplated that a surgeon, after de-bulking the majority of the tumor, may wish to move surgical tool 10 in a clockwise fashion around the margins of the tumor. This may be accomplished, for example, by rotating surgical tool 10 inside the patient's body. At each desired position around the “clock”, the surgeon could take a nibble of tissue and send it to one of the geographically assignable chambers. In one embodiment, these chambers may be aligned circumferentially around the central chamber in a clock-like fashion, as well. So a surgeon taking a nibble at the “1 o'clock” position, for example, would have the tissue core directed to a chamber that can be identified as the 1 o'clock chamber. The number of samples and positions around the clock could be virtually unlimited. Moreover, if multiple rotations around the clock are desired, the receptacle may be replaced with a new receptacle, so that the tissue cores can be separately identifiable. The receptacle could be designed so that it could subsequently be placed into a tissue container having formalin for preserving the samples. The receptacle could be designed to hold the geographic location of each core sample so that its “o'clock” position is identifiable, allowing a pathologist or surgeon to later determine which position(s) around the clock contain abnormal tissue. In one embodiment, the receptacle could be designed to act as a lid for a tissue container, and be attached or screwed on the container, sealing it for later analysis by a pathologist. In yet another embodiment, the receptacle could be previously labeled or automatically labeled by the platform contemplated herein.

One advantage to this method and apparatus is specimens can go directly to lab, rather than being potentially affected by manual removal with tweezers, forceps, needles, etc. Moreover, such a method and apparatus saves the surgical team time during the procedure.

A surgical team (including, for example, a radiologist) may use multiple receptacles, or multiple locations within a single receptacle, showing excision for cure. It is contemplated that a surgeon or radiologist may use three different receptacles or sections. Each of the three sections would incorporate a greater margin of tissue resection. In such a method, it is contemplated that a radiologist may declare a patient resected to tumor free margins if the last two resections—those extending the farthest into the margins—are found to be without cancerous tissue on subsequent histological evaluation. This provides a significant advantage in that a radiologist may be able to perform the entire procedure, with complete resection of all tumorous tissue being the goal, rather than subsequently referring a patient now known to have cancer for surgical lumpectomy in a separate procedure.

Turning to FIG. 28, in one embodiment, receptacle 116 may include a lid 124, an elastomeric gasket 126, a revolving cylinder 128 (having chambers 118, 120 formed therein), and a fluid-receiving base 130. Base 130 may, for example, be connected to a vacuum source to assist with drawing tissue cores into chambers 118, 120. Chambers 118, 120 may also include baskets that hold the tissue cores in place. A retainer 123, a spring washer 125, and a flat washer 127 may secure lid 124 on receptacle 116. A cap 129 may be used to seal the top of receptacle 116.

The receptacle may be a convenient size or shape so that can be placed in a standard hospital laboratory formalin bottle. This provides an advantage of reducing risk of specimens being tampered, damaged mislabeled, technician injury, drop, etc.

It is contemplated that the tissue cores could also be automatically directed to their respective geographically assignable chambers. In this embodiment, surgical tool 10 may be fitted with a weight-positionable disk having a single aperture that moves depending on the rotation of surgical tool 10. When the surgeon rotates, for example, to the 1 o'clock position, the weight-positionable disk (not shown) would rotate within the surgical tool 10, so that its aperture directs any resulting biological material down a specific path associated with the 1 o'clock position. In yet another embodiment, the collection chamber could be directly attached to surgical tool 10, and the weight-positionable disk would direct biological materials into each geographically associated chamber as the surgical tool 10 is rotated to the various positions. The inner cannula rotation, translation, and cutting cycle can be repeated as desired by the surgeon, with a pre-determined pause provided in each stroke so that a surgeon can decide whether to continue.

Additionally, it is contemplated that display unit 48 can be used to allow the operator to choose a specific surgical tool 10 configuration and/or medical procedure. In this embodiment, control circuit 44 will store nominal control parameters for the specific surgical tool 10 and medical procedure, (i.e. a unique recipe for that combination). With each procedure, the operator could indicate (via number, drop-down menu, etc.) what type of procedure and parameters are to be performed. In the alternative, a code, barcode, or other type of identifier could be placed on the surgical tool, to be read by or input into display unit 48 (or in the alternative input into control circuit 44 through some other type of input device). In yet another embodiment, a manual screen could be implemented or provided as an option to allow the operator to adjust the parameters individually, within certain limits, to meet a specific need.

The contemplated control circuit, with its pneumatic operating system, allows surgical tool 10 to be used in any type of imaging environment, including X-Ray, ultrasound, MRI, and mammography. In most cases, the system proposed herein can be applied in a single early-stage outpatient procedure, and most procedures can be completed in a matter of minutes.

Upon completion of the surgical resection, other fluids may be administered by surgical tool 10. For example, as discussed above, chemotherapy, saline, anesthetic, and vasoconstrictor combinations may be administered. Brachytherapy seeds may also be administered. Such fluids or objects may be introduced through the saline line, or may be introduced through a separate conduit, such as in the embodiment shown in FIG. 16. Yet another method of introduction may be through the outer cannula 16, after removing the outer cannula assembly from hub 28. In this embodiment, a surgeon may wish to leave outer cannula 16 in the patient's body, while removing the remainder of surgical tool 10, including the inner cannula 18. This would leave outer cannula 16 to be positioned as an ideal conduit for applications such as fluids, or even surgical markers.

A surgical marker (not shown) may also be introduced through outer cannula 16. In this embodiment, the surgical marker may be a biologically compatible material that is left at the surgical site for later reference. For example, a surgeon may wish to mark the site where the procedure was performed, so that subsequent images may be compared and the precise location of the surgical procedure known. This allows a surgeon to monitor the site for tissue changes or tumor growth.

The surgical marker may be a pre-formed metallic material that forms a certain (e.g. non-linear) shape when it exits outer cannula 16. In one example, this shape may be a ball. However, the surgical marker would optimally be delivered through outer cannula 16 by bending to a substantially straight shape as it is directed through outer cannula 16. In another embodiment, the surgical marker may be formed of a material that takes a different shape when it is introduced to the temperature of a living body. In this embodiment, the surgical marker may be substantially straight when at room temperature, but take a non-linear form when subjected to living body heat. Such a material may be referred to as a “memory alloy” and may comprise, for example, nickel titanium or nitinol.

In another embodiment (not shown), the inner cannula 18 may have a chamfered distal end that is optimized for cutting through tissue. In another embodiment, inner cannula 18 may have a serrated distal end. In yet another embodiment, inner cannula 18 may have an aperture formed in a side wall of the cannula, such cannula capable of acting as a laterally cutting blade.

The surgical platform disclosed herein may be packaged in a convenient package that provides other features for a surgical environment. For example, the package may be a box having magnets that hold the box on top of the control circuit housing described herein. The box may, for example, unfold and have divots or recesses for various tools and attachments. The box may also have a cantilevered tray that extends over the sides of the control circuit, and a drape that covers certain elements of the control circuit (for a more sterile environment). The drape may be, for example, a sterile laminate material that covers the touch screen. The box could advantageously free table space from other places in the operating room.

The surgical tool may have other surgical implements that can replace the inner cannula. For example, a trimmer, a burr, or a drill may be provided as alternative surgical tools. These tools may be advantageous, for example, to orthopedic surgeons.

Surgical tool 10 may also provide tactile or sonic feedback to a surgeon, such as vibrations, sound, or otherwise. This feedback may provide, for example, indications of fibrousness or other information related to the tissue characteristics. It is contemplated that an off-center rotary motor may be used to create some types of tactile feedback.

In one illustrated embodiment, shown in FIG. 25, a specialized hub 112 is used on rotary motor 38. Such a hub 112 causes the six vanes inside rotary motor 38 to be pushed out by air. Pockets or dimples 114 may be formed on the inlet side of the rotor to further promote rotation of the rotor, as might be found in a turbine air motor. Also air can be directed through hub 112 in an angular direction so as to further urge the vanes in a certain direction.

One advantage of the contemplated surgical tool 10 is a design that contemplates only two seals acting as bearings that carry the rotary motor 38 subassembly. This design minimizes the amount of friction in surgical tool 10. The seals may be U-shaped, e.g. “U-cups.”

Another advantage of the contemplated surgical tool 10 is that fluid may be directed over other devices that are carried by surgical tool 10. For example, a camera and/or an ultrasound probe may be carried inside or adjacent to outer cannula 16, and saline may be directed over the camera or probe to clean the camera or probe, and then evacuated by vacuum, the continuous flow ensuring clearer images and/or better performance of the device. In the example of an ultrasound probe, outer cannula 16 may comprise a composite material.

In yet another embodiment, inner cannula 18 may include an opening that can serve as a cutting surface. In this embodiment, inner cannula 18 may be held in the fully advanced position and rotated (e.g. with vacuum applied) such that the opening repeatedly passes by aperture 20, thereby shaving or cutting material adjacent to aperture 20.

In still another embodiment, a sleeve (not shown) may be fitted over the outer cannula. The sleeve may include a plurality of apertures, which would allow for the sleeve to flex. In this embodiment, the sleeve may engage a stopper positioned at the end of outer cannula 16, which would stop axial motion of the sleeve. Further pressure on the sleeve would then cause the sleeve to flex. After being flexed, the sleeve could be rotated and used as a cutter that cuts an ellipsoid- (or other-)shaped tissue ball. Surgical tool 10 could then be operated and suction applied to remove the tissue ball.

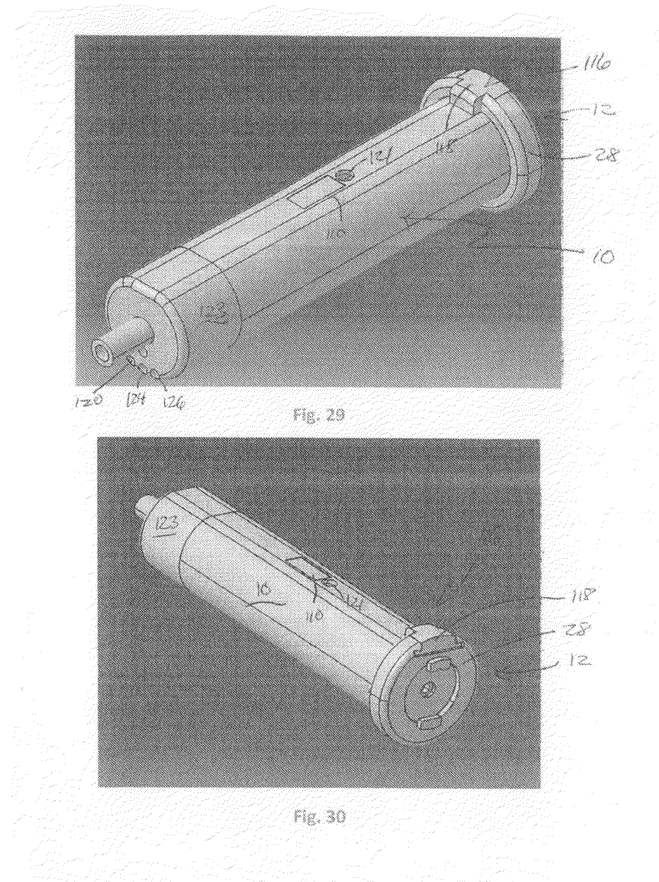

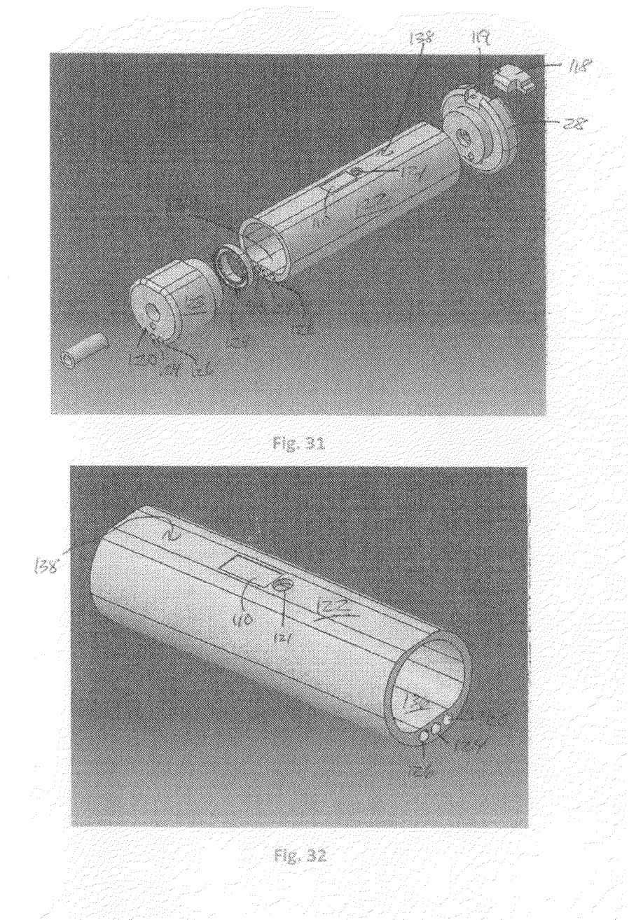

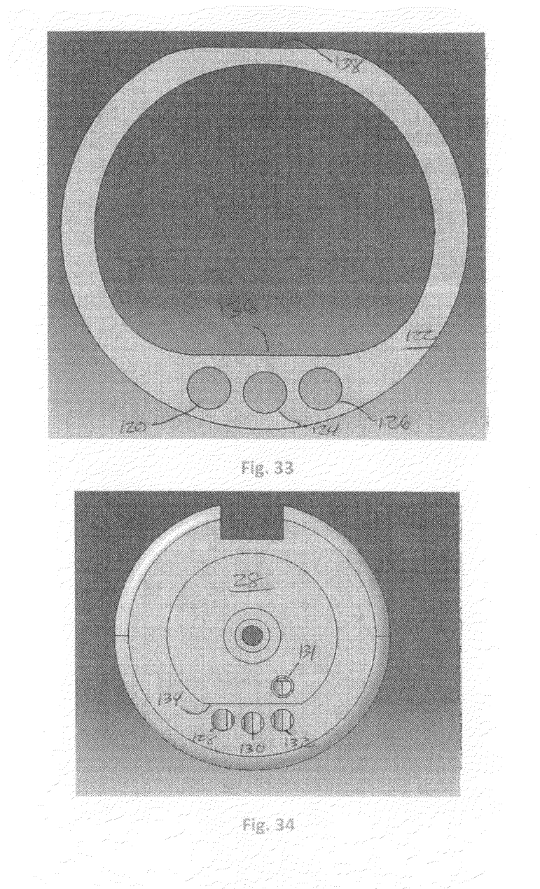

FIGS. 29-31 show another embodiment of an assembled surgical tool 10. According to this embodiment, an activator 116 can be incorporated in surgical tool 10, the activator 116 illustratively comprising a slidable member 118 that moves longitudinally along surgical tool 10. In the illustrated embodiment, slidable member 118 includes a ball plunger poppet valve (not shown) that permits the flow of compressed air through port 119 (shown in FIG. 31) when activated. In this example, an operator may trigger the activator 116 by moving slidable member 118 toward distal end 12 of surgical tool 10. As slidable member 118 is moved, the ball plunger moves off the orifice to allow compressed air in line 120 (shown in FIG. 29 and FIGS. 31-33) to be released. Line 120 is connected to control circuit 44, which senses (e.g. via a low-pressure pressure switch) a drop in pressure. While it is possible to configure activator 116 such that control circuit 44 could sense a rise in pressure via a pressure switch, using an air supply line and a signal line, the described embodiment utilizes only one connection to perform the function.

As can be seen in FIGS. 29-31, activator 116 may be incorporated into hub 28 at distal end 12 of surgical tool 10. Such a position provides the advantage of locating the ball plunger within hub 28, making for easier construction, as well as the advantage of placing activator 116 in a position that is easily accessible by the user's thumb or forefinger. A further advantage is the fact that both right-handed and a left-handed users can easily activate the activator 116.

As illustrated in FIGS. 29-31, hub 28 may also be of larger diameter than the body of surgical tool 10. This allows a user to locate his or her hand and/or thumb on distal end 12 of surgical tool 10, thereby providing a point of leverage and simultaneously a rim that helps a user tactilely locate his or her hand on surgical tool 10, rather than by visual location. Translucent portion 110 is also shown in surgical tool 10, shown in FIGS. 29-32. A hole 121 is also shown in surgical tool 10, for receiving a stopper that provides a stop for the axial movement of rotary motor 38.

Yet another feature of the illustrated embodiment is configuring the rotary motor 38 such that its pneumatic exhaust (not shown) is directed toward pneumatic cutter advancer 40. By directing the exhaust from rotary motor 30 toward cutter advancer 40, cutter advancer 40 functions in part as a muffler for some of the noises created by the exhausting rotary motor 30.

Still a further advantage results from this orientation of the rotary motor 38. By connecting the compressed air source at the distal end of rotary motor 38, and by using the tubular passages disclosed herein, rotary motor 38 is kept away from any tubes or other obstructions that may impede its movement within housing 122. Specifically, the compressed air feed line and/or saline feed line are moved outside of the path of the axially moving rotary motor 38, thereby permitting the unobstructed movement of rotary motor 38. Yet another advantage is housing 122, and therefore surgical tool 10, may be constructed so as to have a smaller length and/or diameter, since less room is required for the movement of rotary motor 38.

As can be seen in FIGS. 31-33, tubular passages or “lines” can be formed in surgical tool housing 122 and end cap 123 such that vacuum pressure, compressed air, and/or liquids may be directed through end cap 123 and housing 122 rather than through external tubes. In the embodiment shown in FIGS. 31-33, line 120 is configured to connect to activator 116, and carries pressurized air that is used by control circuit 44 to sense when activator 116 is activated. Line 124 may be configured to carry saline for supplying the flow of saline between outer cannula 16 and inner cannula 18. Line 126 may be configured to carry compressed air that powers rotary motor 38.

Such a tubular passage construction, such as the embodiment shown in FIGS. 31-33, can be advantageous for a number of reasons. For example, such a construction eliminates or reduces the need for internal or external tubes that are carried along the interior of housing 122 or along the exterior of surgical tool 10. This reduces a potential failure point in the tubes and connections associated therewith, and eliminates the chance that an operator, a motor, or anything else would snag or cut one of the external tubes. The chance of a tube kinking is also eliminated by such a construction. Moreover, as discussed above, this removes any tubes from obstructing the movement of rotary motor 38.

Secondly, assembly is made more efficient by such a construction. Lines 120, 124, and 126 mate with respective ports 128, 130, 132 on hub 28, shown in more detail in FIG. 34. Ports 128, 130, and 132 could be outfitted with nipple fittings, quick-connects, or similar, such that when hub 28 is fitted inside housing 122, a substantially leak-proof seal is created between lines 120, 124, 126 and ports 128, 130, 132. In another embodiment, hub 28 could be affixed with glue, adhesive, or other material to housing 122.

Hub 28 also advantageously includes a flat surface 134, visible in FIG. 34. Such a flat surface 134 mates with housing flat surface 136, visible in FIGS. 31-33. This construction allows hub 28 and housing 122 to be joined with only one orientation—thereby guaranteeing that lines 120, 124, 126 and ports 128, 130, 132 align. End cap 123 and housing 122 align in a similar fashion. FIG. 34 also shows port 131, which is fluidly connected to port 132. Port 131 can be connected to a tube that joins with rotary motor 38, housed within housing 122.

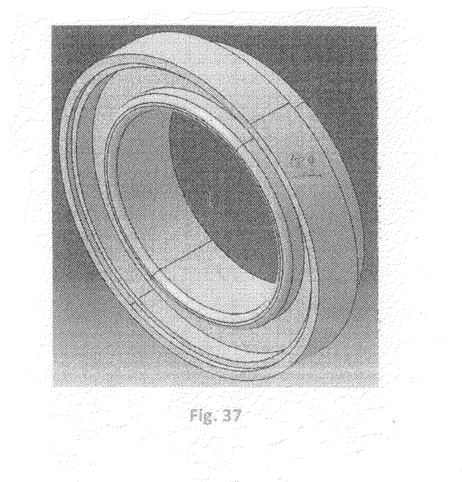

As shown in FIG. 31, and in greater detail in FIG. 37, a cutter advancer holder 129 is configured for placement inside housing 122. Such a cutter advancer holder is configured to hold, for example, the rolling diaphragm embodiment of a cutter advancer disclosed above. Holder 129 will reduce the amount of adhesive that would need to be used to secure the rolling diaphragm. Holder 129 also helps provide a seal around the edge for the compressed air to expand the diaphragm which will help with continually providing control of surgical tool 10.

In one embodiment, the exterior of housing 122 is formed so as to have a non-slip surface. This surface can illustratively be created using bead blasting, or may be formed during the molding process of housing 122.

Housing 122 may also be formed such that it has a flat, indented, or otherwise distinctive surface 138, visible in FIGS. 31-33. Such a distinctive surface is illustratively aligned with aperture 20, and may therefore be formed on the portion of housing 122 that is held by a user's thumb. This distinctive surface allows a user to know without confirming visually the position of aperture 20 in relation to the user's hand.

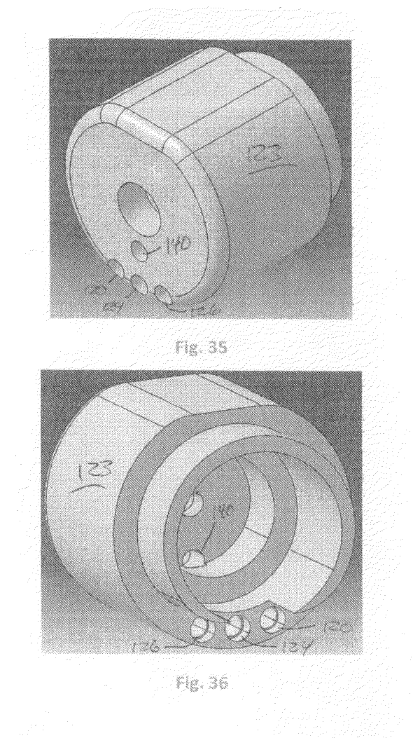

FIGS. 35-36 illustrate perspective views of end cap 123. As can be seen, lines 120, 124, and 126 pass through end cap 123 to join with the lines passing through housing 122. Another passageway, cutter advancer line 140, is formed in end cap 123. Such a cutter advancer line 140 may carry compressed air, vacuum, or both, depending on the type of cutter advancer used.

In yet another embodiment, it is contemplated that a micro- or nano-gyroscope can be placed inside surgical tool 10. Such a micro- or nano-gyroscope can determine coordinates and rotation of the hand wand. An example of a nano-gyroscope that might be capable of incorporation in surgical tool 10 was developed by Tel Aviv University, and is discussed in this article, incorporated herein by reference: http://www2.tau.ac.il/news/engnew.asp?num_new=1909.

It is further contemplated that a micro- or nano-gyroscope that is already imbedded in a separate device, such as in an iPhone 4, may be used in conjunction with surgical tool 10. In this contemplated embodiment, the iPhone 4 (or any other gyroscope-equipped device) would be fixed to surgical tool 10 such that the motion of surgical tool 10 could be detected.

By incorporating a micro- or nano-gyroscope in surgical tool 10, a radiologist may not need to place a guide wire prior to surgical excision. The gyroscope would be capable of detecting where the surgical tool 10 is in relation to the tissue mass to be biopsied. This technology may also be useful in reducing the amount of imaging required prior to resection of the tissue. Still a further advantage that results from this embodiment is such a gyroscope-enabled surgical tool may provide an auto-off feature for control circuit 44 when the surgical tool is set down for a period of time.

In another embodiment of the invention, a magnetic marker is contemplated. By using magnetic material, a marker may be detected by means that are alternative to current imaging. For example, surgical tool 10 may incorporate a metal- or magnet-detecting sensor that determines where the magnetic marker has been placed. Such a feature could also be incorporated with the previously discussed gyroscope. It may also be desirable to encase the magnetic marker in a plastic material so as to prevent biological reactions.

The plastic encapsulate could incorporate micro-bubbles of air or be surface scored or notched thereby enhancing its ability to produce specular reflections with ultrasound examination. Ultrasonic visibility of tissue markers would be advantageous for locating biopsy sites quickly and inexpensively prior to lumpectomy in patients with biopsy-proven breast cancer.

While the disclosure is susceptible to various modifications and alternative forms, specific exemplary embodiments thereof have been shown by way of example in the drawings and have herein been described in detail. It should be understood, however, that there is no intent to limit the disclosure to the particular forms disclosed, but on the contrary, the intention is to cover all modifications, equivalents, and alternatives falling within the spirit and scope of the disclosure as defined by the appended claims.

A plurality of advantages arises from the various features of the present disclosure. It will be noted that alternative embodiments of various components of the disclosure may not include all of the features described yet still benefit from at least some of the advantages of such features. Those of ordinary skill in the art may readily devise their own implementations of a pneumatic circuit that incorporate one or more of the features of the present disclosure and fall within the spirit and scope of the disclosure.

What is claimed is:

1. A medical device comprising:
a control circuit;
a cannula defining a proximal end, a distal end, and a first axis, the cannula having an orifice at the distal end, the distal end being configured for insertion into a body to a point such that the orifice is adjacent to a selected tissue mass;
a cutter positioned such that at least a portion of the cutter extends within the cannula, the cutter defining a second axis coaxial with the first axis and the cutter being movable relative to the cannula; and
a rotary motor coupled to the cutter and configured to rotate the cutter relative to the cannula; the rotary motor defining a third axis in axial alignment with the first and second axes;
wherein the rotary motor and cutter are coupled to the control circuit, and the control circuit is capable of detecting characteristics related to the tissue mass.

2. The medical device of claim 1, wherein the cutter is configured to remove a portion of the selected tissue mass for transportation through the rotary motor.

3. The medical device of claim 1, wherein the tissue mass characteristics include tissue fibrousness and tissue density.

4. The medical device of claim 1, wherein the control circuit records data related to the tissue characteristics and compares such data to previously recorded data.

5. The medical device of claim 1, wherein the medical device further comprises a flow cytometer for detecting characteristics related to the nuclei of the tissue mass.

6. The medical device of claim 1, wherein the medical device further comprises a gyroscope.

7. The medical device of claim 1, wherein the medical device further comprises a housing that houses the rotary motor.

8. The medical device of claim 7, wherein the housing comprises a distinctive surface aligned with the orifice.

9. The medical device of claim 7, wherein the housing comprises an activator that can be triggered by a user.

10. The medical device of claim 9, wherein the activator releases compressed air from a passageway when activated, such released compressed air being sensed by the control circuit.

11. A medical device comprising:
a cannula defining a proximal end, a distal end, and an axis, the cannula having an orifice at the distal end, the distal end being configured for insertion into a body to a point such that the orifice is adjacent to a selected tissue mass;
a cutter coaxially aligned with the cannula and configured to be moved by a cutter advancer;
a motor coupled to the cutter, the cutter defining a passageway that extends through the motor; and
a housing encompassing the cutter advancer and the motor, the housing defining a plurality of tubular passages formed therein.

12. The medical device of claim 11, wherein the tubular passages are capable of carrying compressed air or fluids.

13. The medical device of claim 11, wherein the motor is powered by compressed air that is carried by one of the tubular passages.

14. The medical device of claim 11, further comprising a detachable hub that carries the proximal end of the cannula, the detachable hub having ports formed therein for communication with the plurality of tubular passages.

15. The medical device of claim 14, further comprising an activator coupled to the hub, the activator capable of signaling for the start and stop of the cutter movement.

16. The medical device of claim 11, further comprising a flow cytometer in communication with the cutter passageway, the flow cytometer providing an operator with information relating to the nuclei of tissue cells being resected by the medical device.

17. A medical device comprising:
a cannula defining a proximal end, a distal end, and an axis, the cannula having an orifice at the distal end, the distal end being configured for insertion into a body to a point such that the orifice is adjacent to a selected tissue mass;
a cutter coaxially aligned with the cannula and configured to be moved by a cutter advancer; the cutter advancer being capable of positioning the cutter at a plurality of cutting positions; and
a rotary motor coupled to the cutter, the cutter defining a passageway that extends through the motor.

18. The medical device of claim 17, wherein the cutter advancer can operate such that the cutter has a variable stroke length.

19. The medical device of claim 17, wherein the orifice has a tapered distal end.

20. The medical device of claim 17, further comprising a flow cytometer in communication with the cutter passageway, the flow cytometer providing an operator with information relating to the nuclei of tissue cells being resected by the medical device.

21. The medical device of claim 17, further comprising a marker device having a metallic magnetic center with biocompatible plastic encapsulation altered to produce enhanced visibility with ultrasound examination.
